(19) United States
(12) Patent Application Publication
(10) Pub. No.: US 2010/0130818 A1
(43) Pub. Date: May 27, 2010

(54) CAPSULE-TYPE ENDOSCOPE CAPABLE OF CONTROLLING FRAME RATE OF IMAGE

(76) Inventors: Han Jung, Daejeon (KR); Byung-Hyuk Kim, Daejeon (KR); Yong-Woo Lee, Daejeon (KR); Chul Cha, Daejeon (KR)

Correspondence Address: OSTROLENK FABER GERB & SOFFEN, 1180 AVENUE OF THE AMERICAS, NEW YORK, NY, 100368403, US

(21) Appl. No.: 12/595,991

(22) PCT Filed: Aug. 20, 2008
(86) PCT No.: PCT/KR2008/004823

§ 371 Date: Oct. 15, 2009

(30) Foreign Application Priority Data
Sep. 6, 2007 (KR) 10-2007-0090610

(51) Int. Cl. A61B 1/045

(52) U.S. Cl. 600/109; 600/118

(57) ABSTRACT

A capsule-type endoscope is disclosed. Information related to position and movement of the capsule-type endoscope inserted into a body are checked and a frame rate is controlled according to a digestive organ in which the capsule-type endoscope is located based upon the information, so as to capture every digestive organ inside the body by using one capsule-type endoscope. Also, the frame rate can be controlled according to a moving velocity in each digestive organ, thus to reduce unnecessary power consumption, resulting in a decrease of an operation time of the capsule-type endoscope having a limited battery capacity. In addition, an amount of captured images can be reduced by effectively obtaining images according to the moving velocity in each digestive organ and a time taken by a doctor for a medical examination can be shortened.

### TECHNICAL FIELD

The present invention relates to an endoscope, and more particularly, to a capsule-type endoscope capable of controlling a frame rate of image.

### BACKGROUND ART

A variety of methods for collecting medical information within a human body have been developed.

In general, an endoscope has been used for a method for collecting image information, one of medical information within the human body. Endoscope captures images inside the body and then transmits the captured images to an external device via a communication cable, such as conducting wires and optical fibers. However, if such cable is used in endoscope, even after the endoscope is inserted inside the body, the cable still remains in the mouth (oral cavity), thereby causing a great pain to a patient. In addition, the cable should be manipulated to adjust a region for image capture, thereby causing various side effects, such as damage in the internal organs inside the body, and the like.

In order to solve such problems, Given Imaging Ltd., an Israel-based company has recently developed a capsule-type endoscope, called the “PillCam.” If this capsule-type endoscope is swallowed by a patient, similar to a pill, it may transmit image data related to digestive organs inside the human body captured by an endoscope camera to an external receiver and thus to implement such image data on a monitor.

However, the capsule-type endoscope developed by Given Imaging Ltd., captures images at a fixed frame rate. The speed at which ingesta move within the body depends on the characteristic of each digestive organ within the body. Accordingly, every digestive organ cannot be captured by only one capsule-type endoscope.

That is, a fast frame rate is required to obtain image information related to an internal organ, such as the esophagus where ingesta move fast and frequently, thus to obtain image information therefor without any portion non-captured, whereas a slow frame rate is required in an internal organ, such as small intestine where ingesta move slowly and limitedly, thereby to efficiently obtain image information.

However, since the capsule-type endoscope developed by Given Imaging Ltd., provides a fixed frame rate, an exclusive capsule-type endoscope providing a frame work suitable for each digestive organ has been developed, which causes customers to consume many costs in order to examine every digestive organ.

### DISCLOSURE OF INVENTION

### Technical Solution

Therefore, it is an object of the present invention to control a frame rate of a capsule-type endoscope.

In detail, an object of the present invention is to control a frame rate of a capsule-type endoscope by determining a position of the capsule-type endoscope inserted into a body or information related to a movement of the capsule-type endoscope.

Here, the position of the capsule-type endoscope within the body may be determined by detecting a moving velocity/angular velocity of the capsule-type endoscope. That is, in general, since ingesta fast move in the esophagus but slowly move in small intestine, the moving velocity/angular velocity of the capsule-type endoscope is detected so as to determine the position of the capsule-type endoscope.

Alternatively, the position of the capsule-type endoscope within the body may be determined by calculating a similarity between a plurality of images captured. That is, a low similarity level indicates a fast movement of the capsule-type endoscope and a high similarity level indicates a slow movement thereof. Accordingly, in which digestive organ the capsule-type endoscope is located can be determined.

Alternatively, the position of the capsule-type endoscope within the body may be determined by using an energy level of a received signal. Also, it can be estimated in which organ the capsule-type endoscope is to be located after a certain time elapses according to an average resulting value known through experiments.

In one aspect of the present invention, a capsule-type endoscope capable of controlling a frame rate of image may comprise, a lens adapted to capture an image inside an animal body including a human body, a processor adapted to encode the image captured through the lens to output a signal and control a frame rate according to where the capsule-type endoscope is located within the body, and a transmitting unit adapted to transmit the signal from the processor.

Preferably, the processor may control the frame rate by itself or according to an external control signal.

In case of controlling the frame rate by itself, the processor drives a timer to check an elapsed time after the capsule-type endoscope is located within the body, so as to estimate the position of the capsule-type endoscope, thereby controlling the frame rate.

In case of controlling the frame rate according to the external control signal, the processor encodes a moving velocity of the capsule-type endoscope together with the image information for output, so as to determine the position of the capsule-type endoscope based upon the moving velocity outside the body, thereby controlling the frame rate outside the body.

In another aspect of the present invention, a capsule-type endoscope capable of controlling a frame rate of image may comprise a lens adapted to capture an image inside a digestive organ within an animal body including a human body, a processor adapted to encode the image captured by the lens for output, operate a timer for checking an elapsed time after the capsule-type endoscope is located within the body, and thusly determining in which digestive organ the capsule-type endoscope is located within the body, and to control the frame rate either according to the determined location or according to an external control signal received from the exterior, a transmitting unit adapted to transmit the image signal received from the processor, and a receiving unit adapted to receive the external control signal.

In still another aspect of the present invention, a capsule-type endoscope capable of controlling a frame rate of image may comprise a lens adapted to capture an image inside a body of an animal including human, a processor adapted to encode an image captured by the lens to output the signal and control the frame rate, and a transmitting unit adapted to transmit a signal from the processor. Here, the processor may control the frame rate according to a movement amount of the capsule-type endoscope. The movement amount may correspond to at least one or more of a moving velocity, an angular velocity and a moved distance of the capsule-type endoscope. In order to measure the movement amount, the capsule-type endoscope may further include one or more sensors adapted to measure the movement amount thereof. Also, the capsule-type endoscope may further include a receiving unit adapted to receive an external control signal for controlling the frame rate to provide to the processor.

In an aspect of the present invention, a diagnosis system may include a capsule-type endoscope located in a digestive organ of an animal including human, and adapted to capture an image of the digestive organ at different frame rates while moving in cooperation with the vermiculation of the digestive organ and encode the captured image information to output a signal, a receiver adapted to receive the signal of the image information from the capsule-type endoscope, a signal processing unit adapted to receive the signal of the image information from the receiver, determine where the capsule-type endoscope is located inside the digestive organ of the animal, and output a signal for controlling the frame rate of the capsule-type endoscope, and a transmitter adapted to receive the control signal from the signal processing unit and transmit the control signal to the capsule-type endoscope.

In an aspect of the present invention, a method for controlling a frame rate of image for a capsule-type endoscope may include checking an elapsed time after the capsule-type endoscope is located inside an animal body including a human body, performing a capturing by controlling the frame rate when the checked time corresponds to a preset time, and performing the capturing by controlling the frame rate according to an external control signal when the external control signal for controlling the frame rate is received.

In another aspect of the present invention, a method for controlling a frame rate of image for a capsule-type endoscope may include receiving a signal from the capsule-type endoscope located inside an animal body including a human body, determining where the capsule-type endoscope is located inside the animal body, and controlling the frame rate of the capsule-type endoscope according the determined position.

### ADVANTAGEOUS EFFECTS

The present invention is implemented to determining where a capsule-type endoscope is located within a body and control a frame rate of image according to the determined position, thereby to capture images of every organ inside the body only by using one capsule-type endoscope. Also, the frame rate can be controlled so as to prevent battery consumption, and an amount of the captured images can also be controlled to thusly save time taken by a user to read such images. In addition, by controlling the frame rate of image, more accurate image capturing can be achieved at a portion suspected to be caught by a disease.

### BRIEF DESCRIPTION OF THE DRAWINGS

FIG. 1 is a schematic view illustrating a system including a capsule-type endoscope in accordance with the present invention;

FIG. 2 is a detailed view of the capsule-type endoscope of FIG. 1;

FIG. 3 is a flowchart illustrating an operation of the capsule-type endoscope of FIG. 2;

FIG. 4 is a flowchart illustrating an operation of a signal processing unit of FIG. 1;

FIG. 5 is a flowchart illustrating a detailed operation of the signal processing unit of FIG. 1;

FIG. 6 is an exemplary view illustrating one frame unit of an image captured by the capsule-type endoscope in accordance with the present invention;

FIG. 7 is an exemplary view illustrating several frames of an image captured by a capsule-type endoscope in accordance with the present invention;

FIG. 8 is an exemplary view illustrating a similarity between captured images; and

FIG. 9 is a graph illustrating the similarity between the captured images of FIG. 8.

### MODE FOR THE INVENTION

Hereinafter, the preferred embodiments of the present invention will be described in detail with reference to the accompanying drawings.

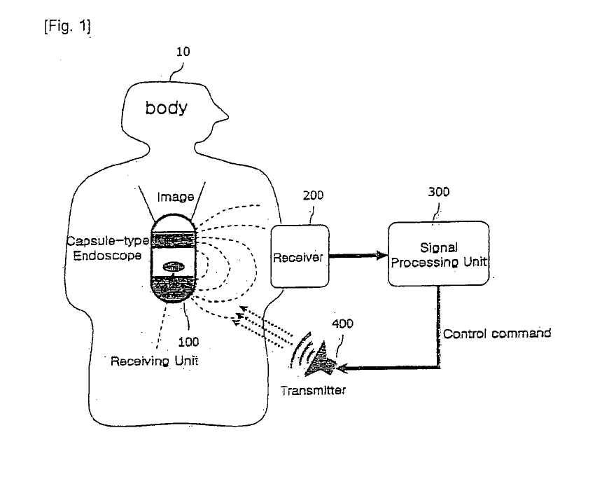

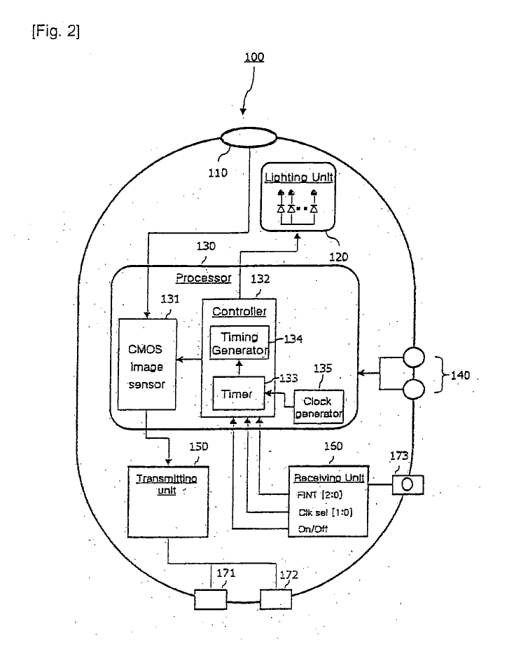

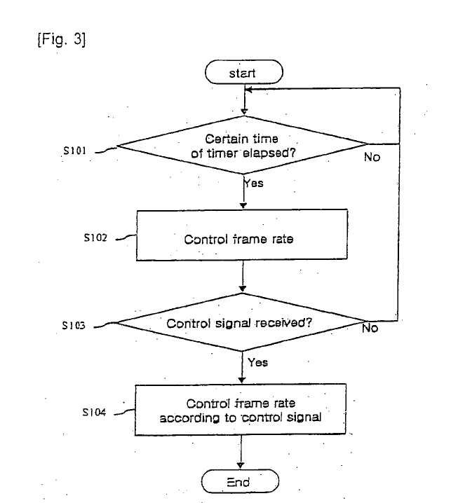

FIG. 1 is a schematic view illustrating a system including a capsule-type endoscope in accordance with the present invention, FIG. 2 illustrates the configuration of the capsule-type endoscope of FIG. 1, and FIG. 3 is a flowchart illustrating an operation of the capsule-type endoscope of FIG. 2.

As shown in FIG. 1, the system according to the present invention may include a capsule-type endoscope 100 located inside a body 10, a receiver 200 attached onto a skin of the body 10 to receive a signal from the capsule-type endoscope 100 for transfer, a signal processing unit 300 for processing the signal transferred from the receiver 200 and outputting a control signal, and a transmitter 400 for transmitting the control signal from the signal processing unit 300 into the capsule-type endoscope 100.

First, a brief description of the present invention is given with reference to FIG. 1 as follows.

The capsule-type endoscope 100 which can be located inside the human or animal body 10, e.g., in a digestive organ, is configured to collect image information or a variety of information (e.g., internal images, pH (potential of hydrogen), temperature or electrical impedance, and the like) and transmit such information to the receiver 200 located on the skin of the body 10 through the body 10. Here, the capsule-type endoscope 100 can either control its frame rate of image by itself according to where it is located inside the body 10, namely, where it is located among esophagus, stomach, small intestine and large intestine, or control the frame rate according to the control signal from the signal processing unit 300. Accordingly, a fast image capturing must be performed in a portion, such as the esophagus, in which the capsule-type endoscope 100 moves fast, thus to obtain image information without any missed portion. On the other hand, a slow image capturing is advantageous in a portion, such as small intestine rarely making a quick motion, in which the capsule-type endoscope 100 moves slowly.

The receiver 200 is configured to transfer the received information to the signal processing unit 300. Here, the receiver 200 can store such signal for a certain time. That is, the receiver 200 may be attached onto the human or animal body as shown in FIG. 1, so as to store the signal received from the capsule-type endoscope 100 for a certain time. Accordingly, without going to the hospital, the human or animal can do an endoscopic examination and an endocrine examination with doing his own work as usual for several hours.

The signal processing unit 300 is configured to process and output the information. The signal processing unit 300 checks a current frame rate of the capsule-type endoscope 100, and confirms where the capsule-type endoscope 100 is located within the body 100, namely, in which of esophagus, stomach, small intestine and large intestine the capsule-type endoscope 100 is located. The signal processing unit 300 then transfers a control command (signal) for controlling the frame rate of the capsule-type endoscope 100 to the capsule-type endoscope 100 via the transmitter 400 according to the confirmation.

The capsule-type endoscope 100 will be described in detail with reference to FIG. 2 as follows. The capsule-type endoscope 100 may include a lens 110, a lighting unit 120 for illuminating light for capturing, a processor 130 for processing an image capturing by the lens 110 and controlling the frame rate of image, one or more sensors 140 for detecting where the capsule-type endoscope 100 is located inside the body 10 or collecting various information related to the inside of the body 10, a transmitting unit 150, a receiving unit 160, transmitting electrodes 171 and 172, and a receiving antenna 173. Here, the one or more sensors 140 may optionally be included or not included.

The lens 110 allows a CMOS image sensor 131 within the processor 130 to capture an image (static image or dynamic image) of an object within the human or animal body 10, for example, in a digestive organ.

The lighting unit 120 is configured to be connected to the processor 130, thus to illuminate light when the lens 110 captures an image inside the body 10 under the control of the processor 130. The lighting unit 120 may be implemented as one or more Light Emitting Diodes (LEDs).

The processor 130 controls the lighting unit 120 to illuminate light with an appropriate intensity of radiation when an image of an object is captured via the lens 110. The processor 130 then encodes the image captured via the lens 110 to output to the transmitting unit 150. The processor 130 can control a frame rate of image for the inside of the body 10 according to where the capsule-type endoscope 100 is located inside the body 10. Such control of the frame rate of image may be achieved by the processor 130 itself or according to an external control signal.

In case of controlling the frame rate of image by itself, the processor 130 drives an internal timer to count a time elapsed after the capsule-type endoscope 110 is located inside the body 10. Then, the processor 130 estimates in which the capsule-type endoscope is to be located inside the body 10 after a preset time elapses, and accordingly can control the frame rate of image. Here, the time of the timer may be set according to an average value resulted from many times of experiments.

Alternatively, in case of controlling the frame rate of image by itself, the processor 130 may obtain a moving velocity/angular velocity from the one or more sensors 140, and check which internal organ corresponds to the obtained moving velocity/angular velocity, so as to enable the control of the frame rate.

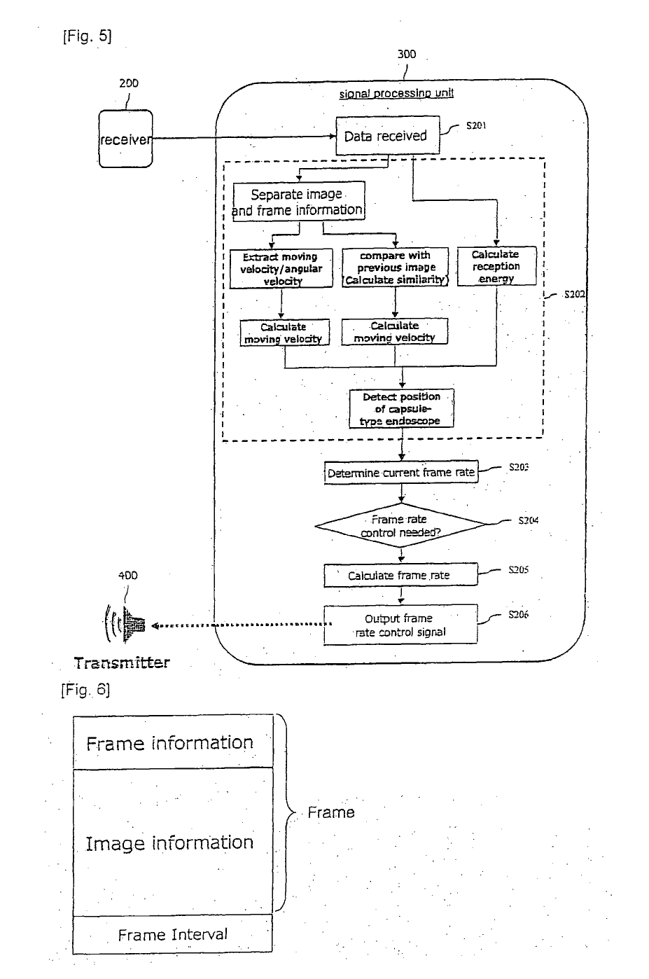

In case of controlling the frame rate of image according to the external control signal, the processor 130 may receive a control signal from the signal processing unit 300 via the receiving unit 160 and controls the frame rate of image according to the control signal. Here, in order for the signal processing unit 300 to recognize where the capsule-type endoscope 100 is located inside the body 10, the processor 130 may encode the information, e.g., the moving velocity/angular velocity, detected by the one or more sensors 140 together with the captured image, thus to output such encoded image to the transmitting unit 150. Here, the information detected by the one or more sensors 140 may be included in a frame header as shown in FIG. 6.

Such processor 130 may concretely include a CMOS image sensor 131, a controller 132, and a clock generator 135.

The CMOS image sensor 131 is configured to encode an image captured by the lens 110 to output a signal, thereby outputting such signal to the transmitting unit 150. Here, the CMOS image sensor 131 may encode information, e.g., moving velocity/angular velocity, detected by the one or more sensors 140 together with the image. The information detected by the one or more sensors 140 may be encoded by being included in the frame header as shown in FIG. 6. The CMOS image sensor 131 may include not only the information from the one or more sensors 140 but also a current frame rate related information in the frame header as shown in FIG. 6, thus to encode together.

The controller 132 may include a timer 133 for counting a time according to a clock from the clock generator 135, and a timing generator 134 for generating a capturing period signal according to the counting of the timer 133. The timing generator 134, as stated above, allows a prediction as to where the capsule-type endoscope 100 is to be located inside the body 10 after a preset time elapses according to the counting of the timer 133, thus to generate a control signal for the capturing at an appropriate frame rate.

On the other hand, the transmitting unit 150 converts the encoded signal through the CMOS image sensor 131 into an electrical signal, so as to apply to two transmitting electrodes 171 and 172 via output lines.

The transmitting electrodes 171 and 172 are configured to come in contact with the inside of the body 10, such that they can generate a potential difference therebetween according to data to be desirably sent, thus to allow a flow of conductive current via the body 10. The current thusly flows from a transmitting electrode having a higher potential to another transmitting electrode having a lower potential via a certain path within the body 10. Here, since the current flowing through the body 10 partially reaches the skin of the body 10, receiving electrodes (not shown) of the receiver 200 attached onto the skin of the body 10 can induce a voltage from the current having reached the skin.

The receiving unit 160 is configured to transfer the control signals received from the transmitter 400 to the processor 130 via the receiving antenna 173. Here, the receiving antenna 173 may be implemented in various manners, such as employing a coil. Here, the control signals may be a FINT signal, a Clk Sel signal, and an On/Off signal of the external control signal. Frame rates according to the control signals may be described as shown in Table 1 below.

TABLE 1

| | Clk sel [1:0] = 00 | | Clk sel [1:0] = 01 | | Clk sel [1:0] = 10 | |
|---|---|---|---|---|---|---|
| | 2.5 Mhz | | 5 Mhz | | 10 Mhz | |
| | operation | | operation | | operation | |
| | Frame | | Frame | | Frame | |
| | Interval | Frame | Interval | Frame | Interval | Frame |
| FINT | Time | Rate | Time | Rate | Time | Rate |
| [2:0] | (ms) | (fps) | (ms) | (fps) | (ms) | (fps) |
| 000 | 2 | 2.5 | 1 | 5.0 | 0.5 | 10.0 |
| 001 | 100 | 2.0 | 50 | 4.0 | 25 | 8.0 |
| 010 | 200 | 1.6 | 100 | 3.2 | 50 | 6.4 |
| 011 | 350 | 1.3 | 175 | 2.6 | 87.5 | 5.2 |
| 100 | 500 | 1.1 | 250 | 2.2 | 125 | 4.4 |
| 101 | 700 | 0.9 | 350 | 1.8 | 175 | 3.6 |
| 110 | 1000 | 0.7 | 500 | 1.4 | 250 | 2.8 |
| 111 | 1500 | 0.5 | 750 | 1.0 | 375 | 2.0 |

As stated above, the configuration of the capsule-type endoscope 100 has been described. Hereinafter, an operation of the capsule-type endoscope 100 will be described.

As shown in FIG. 3, when a certain time according to the counting of the timer 133 elapses (S101), the capsule-type endoscope 100 recognizes to be located at a specific position within the body 10, to control a frame rate accordingly (S102).

Afterwards, the capsule-type endoscope 100 determines whether a control signal has been received from the signal processing unit 300 via the receiving unit 160 (S103). If it is determined that the control signal has not been received, the capsule-type endoscope 100 proceeds back to the step S101.

If it is determined to have received the control signal, then the capsule-type endoscope 100 controls the frame rate according to the control signal (S104).

As described above, the capsule-type endoscope 100 according to the present invention can control the frame rate according to the counting of the timer 133. Also, it can control the frame rate according to the control signal when the control signal is received from the signal processing unit 300.

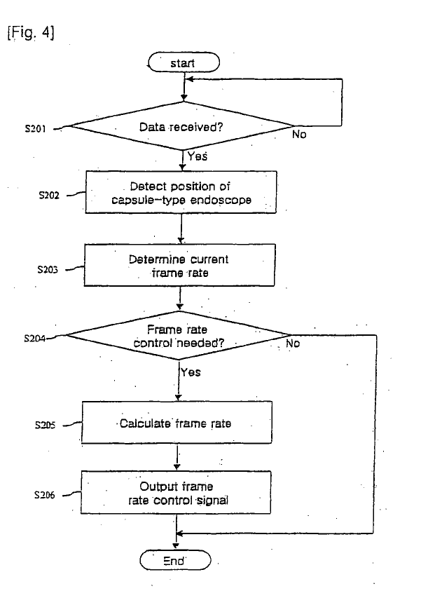

FIG. 4 is a flowchart illustrating an operation of the signal processing unit 300 of FIG. 1.

As shown in FIG. 4, when data is received from the capsule-type endoscope 100 via the receiver 200 (S201), then the signal processing unit 300 according to the present invention determines the position of the capsule-type endoscope 100 (S202).

Then, the signal processing unit 300 checks a current frame rate of the capsule-type endoscope 100 (S203).

The signal processing unit 300 then determines whether the frame rate of the capsule-type endoscope 100 should be controlled (S204).

When the control of the frame rate is needed, the signal processing unit 300 calculates the frame rate (S205), and outputs a control signal via the transmitter 400, thereby transferring the control signal to the capsule-type endoscope 100 (S206).

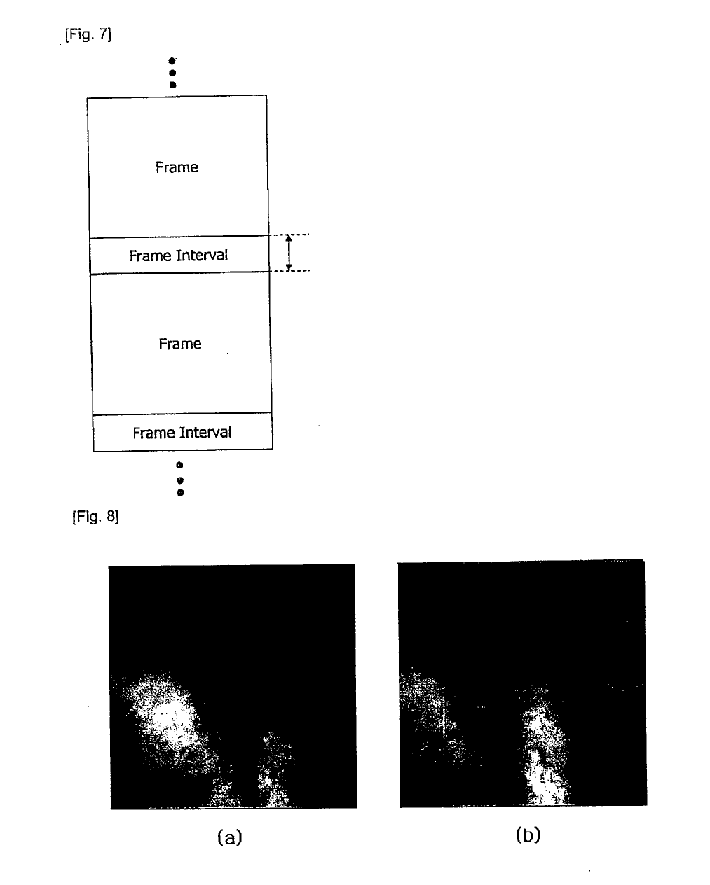

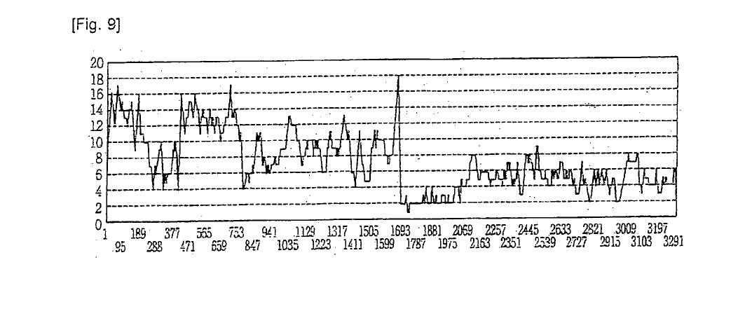

FIG. 5 is a flowchart illustrating a detailed operation of the signal processing unit of FIG. 1, FIG. 6 is an exemplary view illustrating one frame unit of an image captured by the capsule-type endoscope in accordance with the present invention, FIG. 7 is an exemplary view illustrating several frames of an image captured by the capsule-type endoscope in accordance with the present invention, FIG. 8 is an exemplary view illustrating a similarity between captured images, and FIG. 9 is a graph illustrating the similarity between the captured images of FIG. 8.

As shown in FIG. 5, any one of three methods as follows can be employed to check the position of the capsule-type endoscope 100 (i.e., S202).

As a first method, the determining of the position of the capsule-type endoscope 100 (S202) can be achieved such that after separating image information and a frame header from frame-unit data as shown in FIG. 6, information (e.g., information related to the moving velocity/angular velocity of the capsule-type endoscope 100) detected by the aforesaid one or more sensors 140 are extracted from the frame header. That is, as stated above, since the capsule-type endoscope 100 includes the information detected by the one or more sensors 140 (e.g., information related to the moving velocity/angular velocity of the capsule-type endoscope 100) in the frame header of an image corresponding to the inside of the body 10, and accordingly encodes and outputs the frame header together with such information, when extracting the information detected by the one or more sensors 140 from the frame header, the moving velocity of the capsule-type endoscope 100 can be detected. In general, ingesta move in each internal organ (e.g., esophagus, stomach, small intestine, large intestine, and the like) at different velocities. Therefore, once being known of the moving velocity of the capsule-type endoscope 100, it is possible to recognize in which internal organ the capsule-type endoscope 100 is located.

As a second method, the determining of the position of the capsule-type endoscope 100 (S202) can be achieved such that after separating image information and a frame header from frame-unit data as shown in FIG. 6, a similarity between a previous image (e.g., corresponding to FIG. 8(a)) and a current image (e.g., corresponding to FIG. 8(b)) can be checked to detect a moving velocity for detecting where the capsule-type endoscope 100 is located.

Here, the similarity may quantitatively represent how similar the previous image and the current image are. For a high similarity between the previous image and the current image, it denotes the capsule-type endoscope 100 moves fast while for a low similarity therebetween, it denotes the capsule-type endoscope 100 moves slowly. Such similarity may be represented by the following formula, assuming that n^{th} frame is N and n+1^{th} frame is M.

Sum1[n]=ΣAbs(N[x,y])−M[x,y])

Sum2[n]=Σ(N[x,y])−M[x,y])

Similarity[N]=Sum1[n]*100/Sum2[n]

where X denotes a coefficient of a horizontal axis, and y denotes a coefficient of a vertical axis.

Such calculation is performed per every frame. Accordingly, for the faster calculation, 10 frames are averaged, so as to perform the calculation per every 10 frame.

$$AvgSimilarity [ k ] = {\sum }_{n = 1}^{10} Similarity [ k \times 10 + n ] / 10$$

On the other hand, in order to avoid errors, a variety of filters, e.g., Average, Median filter and the like, may be applied.

As such, the calculated similarity can be shown in a graph as shown in FIG. 9.

As a third method, the determining of the position of the capsule-type endoscope 100

(S202) can be achieved by using an energy level of a signal received via the receiver 200. For example, if plural pairs of receiving electrodes of the receiver 200 exist and the plural pairs of electrodes are divided to be attached to several portions on the skin of the body 10, the position of the capsule-type endoscope 100 can be determined by searching for and comparing a database pre-stored with respect to each position, with the signal received via the plural pairs of electrodes.

In addition to the above methods, other methods for determining the position of the capsule-type endoscope may exist, which is obvious to those skilled in the art. Therefore, in order to prevent the concept of the present invention from being obscure, explanation thereof will be omitted. However, even if other methods for determining the position of the capsule-type endoscope are not described, it is apparent to those skilled in the art that the present invention may cover such methods within the claims of the present invention.

In the meantime, for checking the current frame rate (i.e., S203), can be achieved by extracting the current frame rate from the frame header shown in FIG. 6, in case where the capsule-type endoscope 100 includes the current frame rate in the frame header when encoding the image. Also, the checking of the current frame rate (S203) can be implemented either by calculating a time corresponding to a frame interval shown in FIG. 7 or by calculating a time corresponding to one frame. For example, for a frame rate of 5 fps, the frame interval is 1 ms as can be seen in Table 1. For a frame rate of 4 fps, the frame rate is 50 ms. Therefore, the frame rate can be determined by measuring the frame interval.

As the present features may be embodied in several forms without departing from the characteristics thereof, it should also be understood that the above-described exemplary embodiments are not limited by any of the details of the foregoing description, unless otherwise specified, but rather should be construed broadly within its scope as defined in the appended claims, and therefore all changes and modifications that fall within the metes and bounds of the claims, or equivalents of such metes and bounds are therefore intended to be embraced by the appended claims

1. A capsule-type endoscope capable of controlling a frame rate of image comprising:
a lens adapted to capture an image inside an animal body including a human body;
a processor adapted to encode the image captured by the lens to output a signal, and control the frame rate according to a position of the capsule-type endoscope inside the body; and
a transmitting unit adapted to transmit the signal received from the processor.

2. The capsule-type endoscope of claim 1, wherein the processor comprises:
an image sensor adapted to encode the image captured by the lens to output the signal;
a clock generator; and
a controller connected to the image sensor and the clock generator, and adapted to control the frame rate according to a clock from the clock generator.

3. The capsule-type endoscope of claim 1, wherein the processor controls the frame rate by itself or according to an external control signal.

4. The capsule-type endoscope of claim 1, wherein the processor includes a tinier adapted to count an elapsed time after the capsule-type endoscope is located inside the body,
wherein the processor estimates where the capsule-type endoscope is located inside the body by using the timer, thus to control the frame rate.

5. The capsule-type endoscope of claim 1, further comprising one or more sensors adapted to detect information related to the inside of the body.

6. The capsule-type endoscope of claim 5, wherein the one or more sensors detect a velocity or angular velocity of the capsule-type endoscope inside the body, wherein the processor receives the velocity or angular velocity from the one or more sensors, determines where the capsule-type endoscope is located inside the body, and accordingly controls the frame rate.

7. The capsule-type endoscope of claim 5, wherein the one or more sensors detect the velocity or angular velocity of the capsule-type endoscope inside the body, wherein the processor encodes the detected velocity or angular velocity together with the image so as to output a signal.

8. The capsule-type endoscope of claim 7, wherein the processor includes the detected velocity or angular velocity in a frame header of frame data units of the image, so as to encode the same.

9. The capsule-type endoscope of claim 1, further comprising a receiving unit adapted to receive a control signal for controlling the frame rate.

10. The capsule-type endoscope of claim 1, wherein the transmitting unit transmits the signal into the body using a potential difference between two transmitting electrodes.

11. A capsule-type endoscope capable of controlling a frame rate of image comprising:
a lens adapted to capture an image inside a digestive organ of an animal including human;
a processor adapted to encode the image captured by the lens to output a signal, operate a timer for checking an elapsed time after the capsule-type endoscope is located inside the animal, and accordingly determining in which digestive organ the capsule-type endoscope is located inside the animal, and to control the frame rate according to the determined location or an external control signal;
a transmitting unit adapted to transmit the image signal received from the processor; and
a receiving unit adapted to receive the external control signal.

12. The capsule-type endoscope of claim 11, further comprising one or more sensors adapted to detect information related to the inside of the body.

13. The capsule-type endoscope of claim 12, wherein the one or more sensors detect a velocity or angular velocity of the capsule-type endoscope inside the body, wherein the processor encodes the detected velocity or angular velocity together with the image so as to output a signal.

14. The capsule-type endoscope of claim 13, wherein the processor includes the detected velocity or angular velocity in a frame header of frame data units of the image, so as to encode the same.

15. The capsule-type endoscope of claim 11, wherein the transmitting unit transmits the signal into the body using a potential difference between two transmitting electrodes.

16. A capsule-type endoscope capable of controlling a frame rate comprising:
a lens adapted to capture an image inside an animal body including a human body;
a processor adapted to encode the image captured by the lens to output a signal, and control the frame rate of capturing the image; and
a transmitting unit adapted to transmit the signal received from the processor.

17. The capsule-type endoscope of claim 16, wherein the processor controls the frame rate according to a movement amount of the capsule-type endoscope.

18. The capsule-type endoscope of claim 17, further comprising one or more sensors adapted to detect the movement amount of the capsule-type endoscope.

19. The capsule-type endoscope of claim 17, wherein the movement amount denotes at least one or more of velocity, angular velocity and moved distance of the capsule-type endoscope.

20. The capsule-type endoscope of claim 16, further comprising a receiving unit adapted to receive an external control signal for controlling the frame rate and provide the external control signal to the processor.

21. A diagnosis system comprising:
a capsule-type endoscope located in a digestive organ of an animal including human, and adapted to capture an image of the digestive organ at different frame rates while moving in cooperation with the vermiculation of the digestive organ and encode the captured image information to output a signal;
a receiver adapted to receive the signal of the image information from the capsule-type endoscope;
a signal processing unit adapted to receive the signal of the image information from the receiver, determine where the capsule-type endoscope is located inside the digestive organ of the animal, and output a signal for controlling the frame rate of the capsule-type endoscope; and
a transmitter adapted to receive the control signal from the signal processing unit and transmit the control signal to the capsule-type endoscope.

22. The system of claim 21, wherein the signal processing unit measures the magnitude of the signal received from the receiver to determine the position of the capsule-type endoscope.

23. The system of claim 21, wherein the capsule-type endoscope measures a moving velocity thereof inside the digestive organ and encodes the measured moving velocity together with the image to output a signal,
wherein the signal processing unit extracts information related to moving velocity from the signal and determines the position of the capsule-type endoscope based upon the information related to the moving velocity.

24. The system of claim 23, wherein the information related to the moving velocity is included in a frame header portion of frame data units of the image.

25. The system of claim 21, wherein the signal processing unit calculates a similarity between the image and a previous image, determines a moving velocity of the capsule-type endoscope according to the calculated similarity, and detects the position of the capsule-type endoscope according to the determined moving velocity.

26. A method for controlling a frame rate of a capsule-type endoscope comprising:
checking an elapsed time after the capsule-type endoscope is located inside an animal body including a human body;
performing a capturing by controlling the frame rate when the checked time corresponds to a preset time; and
performing the capturing by controlling the frame rate according to an external control signal when the external control signal for controlling the frame rate is received.

27. A method for controlling a frame rate of a capsule-type endoscope comprising:
receiving a signal from the capsule-type endoscope located inside an animal body including a human body;
determining where the capsule-type endoscope is located inside the animal body; and
controlling the frame rate of the capsule-type endoscope according the determined position.

28. The method of claim 27, wherein the determining of the position is configured by measuring the magnitude of the signal received from the capsule-type endoscope.

29. The method of claim 27, wherein the determining of the position is configured by detecting a moving velocity of the capsule-type endoscope according to velocity related information for the capsule-type endoscope included in the signal.

30. The method of claim 27, wherein the determining of the position comprises:
extracting image information from the signal;
calculating a similarity between the image and a previous image;
determining the moving velocity of the capsule-type endoscope according to the similarity; and
detecting the position of the capsule-type endoscope according to the moving velocity.

31. The method of claim 27, further comprising:
checking a current frame rate of the capsule-type endoscope; and
determining whether the current frame rate should be controlled.

32. The method of claim 31, wherein the determining of the frame rate is configured either by measuring an interval between frames of the signal or by measuring a size of one frame.
